(19) United States
(12) Patent Application Publication
(10) Pub. No.: US 2014/0275943 A1
(43) Pub. Date: Sep. 18, 2014

(54) PHOTOACOUSTIC MONITORING TECHNIQUE

(71) Applicant: COVIDIEN LP, Mansfield, MA (US)

(72) Inventors: DongYel Kang, Irvine, CA (US); Bo Chen, Louisville, CO (US); Qiaojian Huang, Broomfield, CO (US); Youzhi Li, Longmont, CO (US)

(73) Assignee: COVIDIEN LP, Mansfield, MA (US)

(21) Appl. No.: 13/840,944

(22) Filed: Mar. 15, 2013

(51) Int. Cl. A61B 5/00; A61B 5/02

(52) U.S. Cl. CPC A61B 5/0095; A61B 5/02
USPC 600/407

(57) ABSTRACT

Various methods and systems for photoacoustic patient monitoring are provided. A photoacoustic system includes a light emitting component that emits one or more wavelengths of light into an interrogation region of a patient and an acoustic detector that detects acoustic energy generated by the interrogation region of the patient in response to the emitted light. The system may be implemented to receive photoacoustic waves from a local vein and artery and determine hemodynamic parameters based on a summed indicator dilution curve representative of both arterial and venous indicator dilution.

### BACKGROUND

The present disclosure relates generally to medical devices and, more particularly, to the use of photoacoustic monitoring techniques for determining physiological parameters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. For example, clinicians may wish to monitor a patient's blood flow to assess cardiac function. In particular, clinicians may wish to monitor a patient's cardiac output. The determination of cardiac output may provide information useful for the diagnosis and treatment of various disease states or patient abnormalities. For example, in cases of pulmonary hypertension, a clinical response may include a decrease in cardiac output.

Accordingly, there are a variety of clinical techniques which may be used for analyzing cardiac output. In one technique, an indicator, such as a dye or saline solution, is injected into a circulatory system of a patient, and information about certain hemodynamic parameters may be determined by assessing the dilution of the indicator after mixing with the bloodstream. However, such techniques involve artery catheters for detecting the dilution of the indicator. Other techniques may involve radioactive indicators that are easier to detect, but these techniques expose the patient to radioactivity and involve expensive detection equipment.

### BRIEF DESCRIPTION

Provided herein are non-invasive photoacoustic techniques that are capable of measuring indicator dilution. Such techniques may involve a photoacoustic sensor and/or an associated monitoring system or methods used in conjunction with such sensors and/or systems.

The disclosed embodiments include a photoacoustic monitoring system that includes a memory storing instructions for receiving a signal from an acoustic detector configured to detect a photoacoustic effect from light emitted into a patient's tissue, wherein the signal is representative of an indicator concentration in an artery and a vein; and determining a physiological parameter based at least in part on the signal. The system also includes a processor configured to execute the instructions.

The disclosed embodiments also include a photoacoustic monitoring system that includes a monitor configured to receive a signal representative of an indicator concentration in an artery and a vein; and determine a physiological parameter based at least in part on the signal. The system also includes a sensor coupled to the monitor that includes a light source configured to emit light into a tissue, wherein the tissue includes an artery and a vein; and a flat or fixed focus ultrasound receiver configured to detect a photoacoustic effect from light emitted into a patient's tissue to generate the signal, wherein a focal length of the flat or fixed focus ultrasound receiver encompasses the artery and the vein.

The disclosed embodiments also include a method with the steps of receiving a signal from a fixed focus or flat acoustic detector configured to detect a photoacoustic effect from light emitted into a patient's tissue, wherein the signal is representative of an indicator concentration in an artery and a vein; and determining a physiological parameter based at least in part on the signal.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and photoacoustic sensor in accordance with an embodiment;

FIG. 2 is a schematic diagram of a photoacoustic sensor inducing a photoacoustic effect in a circulatory system into which an indicator has been injected;

FIG. 3 is a detail view of the photoacoustic sensor of FIG. 2;

FIG. 4 is a flow diagram of a method of determining a hemodynamic parameter in accordance with an embodiment;

FIG. 5 is an example of a photoacoustic dilution curve for a local artery and vein;

FIG. 6 is an example of summed photoacoustic dilution curves for different times;

FIG. 7 shows summed photoacoustic dilution curves for a particular flow rate at different measurement points; and

FIG. 8 shows a comparison between actual flow rate and experimentally determined flow rate for the photoacoustic dilution curves of FIG. 7.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In certain medical contexts it may be desirable to ascertain various localized physiological parameters, such as parameters related to individual blood vessels or other discrete components of the vascular system. Examples of such parameters may include oxygen saturation, hemoglobin concentration, perfusion, and so forth, for an individual blood vessel. In one approach, measurement of such localized parameters is achieved via photoacoustic (PA) spectroscopy. Photoacoustic spectroscopy utilizes light directed into a patient's tissue to generate acoustic waves that may be detected and resolved to determine localized physiological information of interest. In particular, the light energy directed into the tissue may be provided at particular wavelengths that correspond to the absorption profile of one or more blood or tissue constituents of interest. In certain embodiments, the light is emitted as pulses (i.e., pulsed photoacoustic spectroscopy), though in other embodiments the light may be emitted in a continuous manner (i.e., continuous photoacoustic spectroscopy). The light absorbed by the constituent of interest results in a proportionate increase in the kinetic energy of the constituent (i.e., the constituent is heated), which results in the generation of acoustic waves. The acoustic waves may be detected and used to determine the amount of light absorption, and thus the quantity of the constituent of interest, in the illuminated region. For example, the detected ultrasound energy may be proportional to the optical absorption coefficient of the blood or tissue constituent and the fluence of light at the wavelength of interest at the localized region being interrogated (e.g., a specific blood vessel).

When an indicator is injected into a vein in a cardiovascular system, a diluted temporal profile of the indicator may be measured in a downstream artery to estimate the hemodynamic properties. The arterial concentration of the indicator may be used as a marker of cardiac output and is a function of the blood flow as the indicator travels from the vein into the heart and mixes with arterial blood. Accordingly, the indicator concentration in the mixed blood may provide information about hemodynamic properties. Because the indicator dilutes within the bloodstream as it travels through the circulation from the vein to the artery, the measured or estimated arterial concentration of the indicator lags behind the venous concentration at certain points in the dilution curve. In particular, at time zero, i.e., the time of injection, the artery has no indicator while the vein has received a bolus of indicator. Over time, the indicator increases in concentration within the artery and decreases in concentration within the vein. Accordingly, certain indicator dilution techniques attempt to isolate the arterial concentration of the indicator from the venous concentration to avoid introducing error into the measurement. In certain techniques, the measurement of the arterial concentration of the indicator may be a direct measurement (e.g., via an arterial catheter) of the artery.

In contrast to the techniques in which the arterial concentration of an indicator is isolated from or measured separately from the venous concentration, the disclosed embodiments provide a photoacoustic monitoring technique that estimates hemodynamic parameters based on a combined or summed measurement of venous and arterial indicator dilution. Rather than separating the arterial concentration from the venous concentration, the techniques are applied to detected signals that include measurements representative of arterial and venous concentration. For example, when inducing a photoacoustic effect within the tissue, a flat or unfocused acoustic transducer may be used that does not focus only on an artery but instead detects acoustic waves in areas of the tissue that include an artery and vein. Accordingly, because a photoacoustic effect is generated in the artery and vein, the detected acoustic waves are also representative of indicator dilution (e.g., concentration) in the artery and vein. This is also in contrast to other photoacoustic spectroscopy techniques that involve detecting acoustic waves from a tissue location corresponding to only an artery. In one example of such a technique, an ultrasound phased array can be applied for the focusing functionality. The use of targeted focusing components in such techniques is complicated and expensive, in particular because of the cost of the associated ultrasound array.

As provided herein, a fixed-focused and/or flat acoustic transducer (e.g., an ultrasound detector) may be used to detect a photoacoustic effect for determining indicator dilution. In certain embodiments, the use of a fixed-focus acoustic detector reduces complexity and cost of the system components, including sensor and/or associated monitoring components while also maintaining the advantage of a noninvasive technique. Further, using a fixed-focused and/or flat transducer is less sensitive to sensor positioning on the tissue. That is, rather than using sensor positioning to select an artery that is sufficiently spaced apart from a vein to reduce the venous contribution to the signal, the present techniques may be implemented without concern for the presence of venous signal. This in turn reduces operator error or variability. Algorithms applied to photoacoustic indicator dilution curves from the detected summed venous and arterial photoacoustic signal may be used to estimate hemodynamic properties, such as cardiac output, extravascular lung water (EVLW) and other hemodynamic quantities.

With the foregoing in mind, FIG. 1 depicts an example of a photoacoustic monitoring system 8 that may be utilized in determining cardiac output. The system 8 includes a photoacoustic spectroscopy sensor 10 and a monitor 12. Some photoacoustic spectroscopy systems 8 may include one or more photoacoustic sensors 10, as illustrated in FIG. 1, to generate physiological signals for different regions of a patient. For example, in certain embodiments, a single sensor 10 may have sufficient penetration depth to generate physiological signals from deep vessels (e.g., pulmonary artery and/or pulmonary vein). In other embodiments, more than one (e.g., two sensors) sensor 10 may be used to monitor physiological parameters (e.g., oxygen saturation) of more superficial vessels (e.g., the jugular vein and the femoral vein). Further, a system 8 as contemplated may be used in conjunction with other types of medical sensors, e.g., pulse oximetry or regional saturation sensors, to provide input to a multiparameter monitor.

The sensor 10 may emit spatially modulated light at certain wavelengths into a patient's tissue and may detect acoustic waves (e.g., ultrasound waves) generated in response to the emitted light. The monitor 12 may be capable of calculating physiological characteristics based on signals received from the sensor 10 that correspond to the detected acoustic waves. The monitor 12 may include a display 14 and/or a speaker 16 which may be used to convey information about the calculated physiological characteristics to a user. Further, the monitor 12 may be configured to receive user inputs via control input circuitry 17. The sensor 10 may be communicatively coupled to the monitor 12 via a cable or, in some embodiments, via a wireless communication link.

In one embodiment, the sensor 10 may include a light source 18 and an acoustic detector 20, such as an ultrasound transducer. The disclosed embodiments may generally describe the use of continuous wave (CW) light sources to facilitate explanation. However, it should be appreciated that the photoacoustic sensor 10 may also be adapted for use with other types of light sources, such as pulsed light sources, in other embodiments. In certain embodiments, the light source 18 may be associated with one or more optical fibers for conveying light from one or more light generating components to the tissue site.

For example, in one embodiment the light source 18 may be one, two, or more light emitting components (such as light emitting diodes) adapted to transmit light at one or more specified wavelengths. In certain embodiments, the light source 18 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1100 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1100 nm) may be used. In one embodiment, the selected wavelengths of light may penetrate between 1 mm to 3 cm into the tissue of the patient. In certain embodiments, the selected wavelengths may penetrate through bone (e.g., skull) of the patient.

One problem that may arise in photoacoustic spectroscopy may be attributed to the tendency of the emitted light to diffuse or scatter in the tissue of the patient. As a result, light emitted toward an internal structure or region, such as a blood vessel, may be diffused prior to reaching the region so that amount of light reaching the region is less than desired. Therefore, due to the diffusion of the light, less light may be available to be absorbed by the constituent of interest in the target region, thus reducing the ultrasonic waves generated at the target region of interest, such as a blood vessel. To increase the precision of the measurements, the emitted light may be focused on an internal region of interest by modulating the intensity and/or phase of the illuminating light.

Accordingly, an acousto-optic modulator (AOM) 24 may modulate the intensity of the emitted light, for example, by using LFM techniques. The emitted light may be intensity modulated by the AOM 24 or by changes in the driving current of the LED emitting the light. The intensity modulation may result in any suitable frequency, such as from 1 MHz to 10 MHz or more. Accordingly, in one embodiment, the light source 18 may emit LFM chirps at a frequency sweep range approximately from 1 MHz to 5 MHz. In another embodiment, the frequency sweep range may be of approximately 0.5 MHz to 10 MHz. The frequency of the emitted light may be increasing with time during the duration of the chirp. In certain embodiments, the chirp may last approximately 0.1 second or less and have an associated energy of a 10 mJ or less, such as between 1 μJ to 2 mJ, 1-5 mJ, 1-10 mJ. In such an embodiment, the limited duration of the light may prevent heating of the tissue while still emitting light of sufficient energy into the region of interest to generate the desired acoustic waves when absorbed by the constituent of interest.

Additionally, the light emitted by the light source 18 may be spatially modulated, such as via a modulator 26. For example, in one embodiment, the modulator 26 may be a spatial light modulator, such as a Holoeye® LC-R 2500 liquid crystal spatial light modulator. In one such embodiment, the spatial light modulator may have a resolution of 1024×768 pixels or any other suitable pixel resolution. During operation, the pixels of the modulator 26 may be divided into subgroups (such as square or rectangular subarrays or groupings of pixels) and the pixels within a subgroup may generally operate together. For example, the pixels of a modulator 26 may be generally divided into square arrays of 10×10, 20×20, 40×40, or 50×50 pixels. In one embodiment, each subgroup of pixels of the modulator 26 may be operated independently of the other subgroups. The pixels within a subgroup may be operated jointly (i.e., are on or off at the same time) though the subgroups themselves may be operated independently of one another. In this manner, each subgroup of pixels of the modulator 26 may be operated so as to introduce phase differences at different spatial locations within the emitted light. That is, the modulated light that has passed through one subgroup of pixels may be at one phase and that phase may be the same or different than the modulated light that has passed through other subgroups of pixels, i.e., some segments or portions of the modulated light wavefront may be ahead of or behind other portions of the wavefront. In one embodiment, the modulator 26 may be associated with additional optical components (e.g., lenses, reflectors, refraction gradients, polarizers, and so forth) through which the spatially modulated light passes before reaching the tissue of the patient 22.

In one example, the acoustic detector 20 may be one or more ultrasound transducers suitable for detecting ultrasound waves emanating from the tissue in response to the emitted light and for generating a respective optical or electrical signal in response to the ultrasound waves. For example, the acoustic detector 20 may be suitable for measuring the frequency and/or amplitude of the ultrasonic waves, the shape of the ultrasonic waves, and/or the time delay associated with the ultrasonic waves with respect to the light emission that generated the respective waves. In one embodiment an acoustic detector 20 may be an ultrasound transducer employing piezoelectric or capacitive elements to generate an electrical signal in response to acoustic energy emanating from the tissue of the patient, i.e., the transducer converts the acoustic energy into an electrical signal.

In one embodiment, the acoustic detector 20 may a flat or fixed-focus detector. That is, the acoustic detector 20 may be configured to detect acoustic waves within a particular focal region that is not configured to be changed once the sensor 10 is applied. This is in contrast to variable focus detectors that can be controlled to change the focal area. A fixed-focus detector may be mechanically moved (e.g., by an operator) to move the detecting area of the sensor relative to the tissue. Further, the acoustic detector 20 may be a flat transducer.

In one implementation, the acoustic detector 20 may be a low finesse Fabry-Perot interferometer mounted on an optical fiber. In such an embodiment, the incident acoustic waves emanating from the probed tissue modulate the thickness of a thin polymer film. This produces a corresponding intensity modulation of light reflected from the film. Accordingly, the acoustic waves are converted to optical information, which is transmitted through the optical fiber to an upstream optical detector, which may be any suitable detector. In some embodiments, a change in phase of the detected light may be detected via an appropriate interferometry device which generates an electrical signal that may be processed by the monitor 12. The use of a thin film as the acoustic detecting surface allows high sensitivity to be achieved, even for films of micrometer or tens of micrometers in thickness. In one embodiment, the thin film may be a 0.25 mm diameter disk of 50 micrometer thickness polyethylene terepthalate with an at least partially optically reflective (e.g., 40% reflective) aluminum coating on one side and a mirror reflective coating on the other (e.g., 100% reflective) that form the mirrors of the interferometer. The optical fiber may be any suitable fiber, such as a 50 micrometer core silica multimode fiber of numerical aperture 0.1 and an outer diameter of 0.25 mm.

The photoacoustic sensor 10 may include a memory or other data encoding component, depicted in FIG. 1 as an encoder 28. For example, the encoder 28 may be a solid state memory, a resistor, or combination of resistors and/or memory components that may be read or decoded by the monitor 12, such as via reader/decoder 30, to provide the monitor 12 with information about the attached sensor 10. For example, the encoder 28 may encode information about the sensor 10 or its components (such as information about the light source 18 and/or the acoustic detector 20). Such encoded information may include information about the configuration or location of photoacoustic sensor 10, information about the type of lights source(s) 18 present on the sensor 10, information about the wavelengths, light wave frequencies, chirp durations, and/or light wave energies which the light source(s) 18 are capable of emitting and the properties and/or the focal range of the acoustic detector 20, information about the nature of the acoustic detector 20, and so forth. In certain embodiments, the information also includes a reference linear frequency modulation (LFM) chirp that was used to generate the actual LFM emitted light. This information may allow the monitor 12 to select appropriate algorithms and/or calibration coefficients for calculating the patient's physiological characteristics, such as the amount or concentration of a constituent of interest in a localized region, such as a blood vessel.

In one implementation, signals from the acoustic detector 20 (and decoded data from the encoder 28, if present) may be transmitted to the monitor 12. The monitor 12 may include data processing circuitry (such as one or more processors 32, application specific integrated circuits (ASICS), or so forth) coupled to an internal bus 34. Also connected to the bus 34 may be a RAM memory 36, a ROM memory 38, a speaker 16 and/or a display 14. In one embodiment, a time processing unit (TPU) 40 may provide timing control signals to light drive circuitry 42, which controls operation of the light source 18, such as to control when, for how long, and/or how frequently the light source 18 is activated, and if multiple light sources are used, the multiplexed timing for the different light sources.

The TPU 40 may also control or contribute to operation of the acoustic detector 20 such that timing information for data acquired using the acoustic detector 20 may be obtained. Such timing information may be used in interpreting the acoustic wave data and/or in generating physiological information of interest from such acoustic data. For example, the timing of the acoustic data acquired using the acoustic detector 20 may be associated with the light emission profile of the light source 18 during data acquisition. Likewise, in one embodiment, data acquisition by the acoustic detector 20 may be gated, such as via a switching circuit 44, to account for differing aspects of light emission. For example, operation of the switching circuit 44 may allow for separate or discrete acquisition of data that corresponds to different respective wavelengths of light emitted at different times.

The received signal from the acoustic detector 20 may be amplified (such as via amplifier 46), may be filtered (such as via filter 48), and/or may be digitized if initially analog (such as via an analog-to-digital converter 50). The digital data may be provided directly to the processor 32, may be stored in the RAM 36, and/or may be stored in a queued serial module (QSM) 52 prior to being downloaded to RAM 36 as QSM 52 fills up. In one embodiment, there may be separate, parallel paths for separate amplifiers, filters, and/or A/D converters provided for different respective light wavelengths or spectra used to generate the acoustic data.

The data processing circuitry, such as processor 32, may derive one or more physiological characteristics based on data generated by the photoacoustic sensor 10. For example, based at least in part upon data received from the acoustic detector 20, the processor 32 may calculate the amount or concentration of a constituent of interest in a localized region of tissue or blood using various algorithms. In certain embodiments, the processor 32 may calculate one or more hemodynamic properties using signals obtained from one or more sensors 10. In one embodiment, the processor 32 may calculate one or more of cardiac output, total blood volume, extravascular lung water, intrathoracic blood volume, and/or macro and microvascular blood flow from signals obtained from a signal sensor 10. In certain embodiments, these algorithms may use coefficients, which may be empirically determined, that relate the detected acoustic waves generated in response to emitted light waves at a particular wavelength or wavelengths to a given concentration or quantity of a constituent of interest within a localized region.

In one embodiment, processor 32 may access and execute coded instructions, such as for implementing the algorithms discussed herein, from one or more storage components of the monitor 12, such as the RAM 36, the ROM 38, and/or a mass storage 54. Additionally, the RAM 36, ROM 38, and/or the mass storage 54 may serve as data repositories for information such as templates for LFM reference chirps, coefficient curves, and so forth. For example, code encoding executable algorithms may be stored in the ROM 38 or mass storage device 54 (such as a magnetic or solid state hard drive or memory or an optical disk or memory) and accessed and operated according to processor 32 instructions using stored data. Such algorithms, when executed and provided with data from the sensor 10, may calculate one or more physiological characteristics as discussed herein (such as the type, concentration, and/or amount of an indicator). Once calculated, the physiological characteristics may be displayed on the display 14 for a caregiver to monitor or review. Additionally, the calculated physiological characteristics, such as the hemodynamic parameters, may be sent to a multi-parameter monitor for further processing and display. Alternatively, the processor 32 may use the algorithms to calculate the cardiac output, and the cardiac output may be displayed on the display 14 of the monitor 12.

The photoacoustic dilution curve in an artery is the result of indicator concentration variation (i.e. indicator dilution). For human body, the indicator instantaneously injected into the right atrium is diluted in a vascular system. In the vascular system, the local circulatory system may be used as a measurement site for assessing indicator dilution. For example, a photoacoustic sensor may be applied to the temporal artery and vein. For system in which the arterial contribution is assessed, the indicator dilution curve on the local vein can be calculated from the convolution between the artery dilution curve and a dilution point spread function (DPSF) of the local circulatory system. The DPSF is the dilution on the local vein for the unit instantaneous injection onto the local artery.

A focused detector used in conjunction with a photoacoustic monitoring system may yield an enhanced signal from the focused target due to phase matching and avoidance of venous damping by phase mismatching. However, such implementations typically use an ultrasound array that is relatively expensive and complicated to control. The present techniques incorporate a flat or fixed focus acoustic detector that measures photoacoustic indicator dilution from the artery and vein together. As shown in FIG. 2, when an indicator solution (e.g., an isotonic indicator) is injected (arrow 60) by a central venous catheter, the indicator travels via the central vein 62 into the vascular system through various compartments (e.g., the right heart and left heart and into the local artery 86, and then circulate via local vein 84 back to central vein 62. The photoacoustic sensor 10 applied to the skin at a location corresponding to a local vein 84 and a local artery 86 shines light 70 into the tissue and receives returned acoustic waves 80 corresponding to both the local vein 84 and the local artery. From the received signal, indicator dilution curves may be generated. In particular, photoacoustic dilution curves are present in both artery and vein. The photoacoustic sensor 10 measures the summed photoacoustic dilution curve with some time difference.

FIG. 3 is a detail view of the photoacoustic sensor 10 applied to the tissue in FIG. 2. The light 70 induces a photoacoustic effect from the tissue that is experienced by both the local vein 84 and the local artery 86. Accordingly, the generated acoustic waves 80 include a component 90 from the vein 84 and a component 92 from the artery 86. Depending on the phase mismatch, the summed generated waves 80 may be partially damped. The acoustic detector 20 detects the total generated waves 80 from both the local vein 84 and the local artery 86. The local artery 84 and the local vein 86 may be located at the same or different depths. Further, there may be multiple veins located around an artery at the targeted site. While in the depicted embodiment the local vein 84 is deeper than the local artery 86, it should be understood that the received signal may represent a photoacoustic signal from one or more local veins 84 that are deeper than, shallower than, and/or parallel to (e.g., approximately the same depth as) the local artery 86.

As provided herein, the use of a flat or fixed focus (e.g., a long focus) detector 20 facilitates signal acquisition from an artery and other nearby vessels such as veins without requiring differentiation between the arterial and the venous signals. In this manner, the sensor 10 may be applied with less concern as to possible nearby veins that may influence the signal. Another advantage of a flat or fixed focus detector 20 is elimination of focusing steps that are involved in focusing on a target artery. Depending on the sensor location, the patient's size, and medical condition, the location and depth of a target artery and its position relative to a vein may vary. For example, a femoral artery may be less than 2 cm below the surface of the skin for some patients, while in an obese or larger patient, the femoral artery may be 5-7 cm below the skin. Further, certain locations of the temporal artery may be relatively superficial, i.e., close to the skin surface. In one embodiment, the photoacoustic sensor 10 as provided may be configured for a particular monitoring site, such as the head, neck, thigh, or arm. Accordingly, the focal length or focal depth of the acoustic detector 20 may be selected according to the desired measurement site. Accordingly, the fixed focus acoustic detector 20 as provided may have a longer focal range, facilitating detection of the venous and arterial components of the generated acoustic waves. In one embodiment, the focal length of the acoustic detector 20 is about 7 cm or less, about 5 cm or less, or about 2 cm or less. In particular embodiments, the focal length of the acoustic detector 20 is between 2-7 cm, 2-5 cm, or 0.1 cm-2 cm. Further, the acoustic detector may be configured to detect waves at an appropriate frequency. For example, in one embodiment, the acoustic detector has a central frequency of 1 MHz or in a range of 1-5 MHz. In particular implementations, the encoder 28 may include stored information regarding the sensor configuration, such as the focal length and/or frequency band of the acoustic detector 20.

The system 8 as provided may be used only in conjunction with a flat or fixed focus acoustic or detector. That is, the flat or fixed focus acoustic detector 20 as provided may be the only type of detector used with the system 8. In other embodiments, the system 8 may be configured to be used with a flat or fixed focused acoustic detector as well as an acoustic detector 20 with a variable focus. For example, a less expensive photoacoustic sensor 10 with a flat or fixed-focus acoustic detector 20 may be a first choice sensor. If the indicator dilutions curves are not able to be resolved with the flat or fixed focus acoustic detector 20, a photoacoustic sensor 10 with a variable focus acoustic detector 20 may be applied to the patient. Accordingly, the system 8 may include different types of photoacoustic sensors 10 as part of a kit.

FIG. 4 is a process flow diagram illustrating a method for determining a hemodynamic parameter in conjunction with the photoacoustic sensors 10 and systems 8 as provided. The method is generally indicated by reference number 100 and includes various steps or actions represented by blocks. It should be noted that the method 100 may be performed as an automated procedure by a system, such as system 10. Further, certain steps or portions of the method may be performed by separate devices. For example, a first portion of the method 100 may be performed by a caregiver, while a second portion of the method 100 may be performed by a sensor and/or monitor 12 operating under processor control and in response to signals received from the sensor 10. In addition, insofar as steps of the methods disclosed herein are applied to the received signals, it should be understood that the received signals may be raw signals or processed signals. That is, the methods may be applied to an output of the received signals.

In certain embodiments, the method 100 begins with application of the photoacoustic sensor 10 to the patient at step 102. At step 104, an appropriate indicator is injected or otherwise supplied to the patient. In one embodiment, the caregiver may provide an input to the monitor 12 to indicate the indicator injection time point. In certain embodiments, the indicator may be provided as two or more indicators, which may be applied sequentially, according to the desired measured parameter. In one embodiment, the indicator is an isotonic indictor. At step 106, a monitoring device, such as the monitor 12, receives an acoustic detector signal from the photoacoustic sensor 10 that is representative of detected photoacoustic waves in the tissue from a local artery and vein. The desired hemodynamic parameter may be determined at step 108 from the received signal from the acoustic detector 20 and an indicator of the hemodynamic parameter may be provided by the monitor 12 at step 132.

FIGS. 5 and 6 are examples of simulated photoacoustic dilution curves on local artery and vein and summed photoacoustic dilutions. For convenience, the indicator injection time is set to 0 and photoacoustic noise is not considered in this simulation. The parameters are V_{RH}=120 cc, V_{L}=550 cc, V_{LH}=200 cc, V_{T1}=40 cc, V_{T2}=100 cc, and flow rate F=2 liter/min. The term Δt means a time delay of the DPSF on the local vein for the instantaneous injection on the local artery (Δt=5 seconds in FIG. 5), which depends on a flow rate and local circulatory system in a clinical example. As shown in FIG. 6, two peaks of artery and vein dilution curves are not clearly observed in the summed photoacoustic dilution curves with even Δt=15 seconds. Applying algorithms for estimating cardiac output from a single photoacoustic dilution curve, such as those provided herein, to the simulated summed dilution curves estimates a flow rate correctly. The correct estimation is shown with different flow rates and each blood volumes. This holds for even different background photoacoustic signals of the local artery and vein and much longer Δt estimated flow rates are exactly the same as the expected ones based on the experimental model. These simulation results indicate that measuring the summed photoacoustic dilution using fixed-focused or flat ultrasound receivers may be used to estimate cardiac output without removing the venous contribution from the summed photoacoustic indicator dilution curves.

FIG. 7 shows experimental results using an ultrasound receiver to detect generated ultrasound waves with a 1 MHz central frequency and a 0.8 inches (2.032 cm) focal length. The ultrasound receiver was used to measure summed photoacoustic dilutions from two pig blood tubes, sized with 1.47 mm and 1.91 mm of inner and outer diameters, respectively. The two tubes, representing arterial and venous flow, were placed close to one another and covered by the largely defocused receiver so that the photoacoustic dilution curves from each tube are measured together. For the measured summed photoacoustic indicator dilution curves in FIG. 7, the tube length differences between artery and vein measurement points was 40 cm and 80 cm, where Δt was up to 6 seconds for the fastest flow rate; 52.5 ml/min for a 28 flow speed number. The characteristics of experimentally measured summed photoacoustic dilution curves in FIG. 7 are very similar to cases of Δt=5 and 10 seconds in FIG. 6.

FIG. 8 shows the estimated flow rates by applying the cardiac output algorithm to the measured summed photoacoustic dilution curves. Although slightly overestimated, the trends are in good agreement with the actual flow rates. The overestimation is possibly caused by photoacoustic signals insensitive to the indicator concentration. In conclusion, noninvasively estimating cardiac output in photoacoustic imaging may be implemented with fixed-focused and/or flat ultrasound receivers. This approach can remove the complexity and cost in using an ultrasound array.

In certain implementations, the disclosed embodiments may be used in conjunction with suitable noise reduction techniques, such as those provided in “PHOTOACOUSTIC MONITORING TECHNIQUE WITH NOISE REDUCTION,” to Dongyel Kang et al., assigned to Covidien LP, and filed on Mar. 15, 2013, which is hereby incorporated by reference in its entirety herein. Further, as discussed herein, the disclosed techniques may be used to calculate physiological parameters, such as hemodynamic parameters. Accordingly, the disclosed embodiments may use the summed acoustic detector signal as an input to hemodynamic parameter algorithms where the photoacoustic detector signal or the photoacoustic signal PA is denoted as an input. For example, the summed photoacoustic detector signal may be used to determine cardiac output. In one embodiment, if V_{It}, the amount of an isotonic solution, is instantaneously injected at t=0 (i.e. the time of starting the injection is set to zero), the blood flow rate at the outlet point for the PA measurement is:

$$F = \frac{{V}_{It}}{{\int }_{0}^{∞} \frac{{V}_{I} ( t )}{V} d t} \qquad ( 5 )$$

where V and VI(t) are blood volume and isotonic volume rates during the unit time interval, Δt, respectively, in the sectional surface at the outlet point. Equation (5) indicates that the whole saline indicator passes through the outlet sectional surface after the injection. A photoacoustic signal is proportional to an absorption coefficient, μₐ of artery blood that is also proportional to a total hemoglobin concentration, C_{tHb} in the blood vessel. Therefore, the background photoacoustic signal before the indicator injection can be

$${PA}_{b} = K \frac{{tHb}_{b}}{V} \qquad ( 6 )$$

where tHb_{b} is the total hemoglobin in the unit blood volume V associated with Δt. K is the conversion coefficient from C_{tHb} to a photoacoustic signal, which is assumed as constant during the indicator dilution measurement. K contains also other photoacoustic systematic effects, such as fluence in photoacoustic imaging. At the outlet point after the injection, the total hemoglobin in tHb_{b} is decreased due to the added portion of the isotonic solution, V_{I}(t). For this situation, the measured PA signal variation per Δt can be described as

$$PA ( t ) = K \cdot {c}_{tHb} ( t ) = K \frac{{tHb}_{m} ( t )}{{V}_{m} ( t ) + {V}_{I} ( t ) ′} \qquad ( 7 )$$

where Vₘ(t)+V_{I}(t)=V. Since V_{I}(t) is added to the total volume, V, the total hemoglobin in V, tHbₘ(t) is smaller than tHb_{b}. However, the hemoglobin concentration in pure blood (i.e. the blood without the isotonic solution) is not changed by the injection, so

$$\frac{{tHb}_{b}}{V} = \frac{{tHb}_{m} ( t )}{{V}_{m} ( t )} \qquad ( 8 )$$

By substituting Eq. (8) to Eq. (7), the measured photoacoustic signal, PA(t) is

$$PA ( t ) = K \frac{{V}_{m} ( t ) \cdot {tHb}_{b}}{{V}^{2}} = K \frac{[ V - {V}_{I} ( t ) ] \cdot {tHb}_{b}}{{V}^{2}} \qquad ( 9 )$$

Considering Eq. (6), Eq. (9) is further developed to

$$PA ( t ) = {PA}_{b} [ 1 - \frac{{V}_{I} ( t )}{V} ] . \qquad ( 10 )$$

Here, it is assumed that PA_{b} is stationary in time. Integrating both sides of Eq. (10) in time derives the blood flow rate as

$$F = \frac{{V}_{It}}{[ {\int }_{0}^{∞} d t - {\int }_{0}^{∞} \frac{PA ( t )}{{PA}_{b}} ]} \qquad ( 11 )$$

where Eq. (5) is applied to the derivation of Eq. (11). Since a photoacoustic signal measured at the outlet point is decreased due to the isotonic injectate, the denominator of Eq. (11) indicates the area between the photoacoustic dilution curve and the normalized baseline, 1. The normalization in the integration of Eq. (11) is obtained during the derivation process, which is from that the photoacoustic signal is proportional to the inverse of the amount of an isotonic solution. Assumptions in other techniques may include (1) The system is “stationary” (flow F and the system configuration do not change with time), (2) indicator and fluid particles behave exactly the same, (3) indicator and fluid particles have identical transit time distributions, (4) each particle entering the system will leave it after a finite time, (5) there is no recirculation, and (6) dead volumes, meaning volumes that can be entered neither by flowing fluid particles nor by indicator particles. For the photoacoustic indicator dilution technique, several of these assumptions are removed (e.g., (2), (3), (4), (6) and (7)), leaving assumptions (1) and (5). Accordingly, the disclosed techniques also may improve the potential error sources by removing a number of assumptions. For thermodilution techniques, the temperature variation of injectates before the injection and unexpected loss of indicator temperature after injection are additional error sources that are also not associated with the disclosed techniques.

In another embodiment, the disclosed techniques may be used for estimating the extravascular lung water (EVLW) from double indicator dilution curves. For this double indicator technique, two indicators of isotonic and hypertonic bolus are injected into the venous circulation in series. The injected isotonic indicator passes through a vascular system without the interaction with lung tissues. The photoacoustic signal monitoring the variation of the isotonic solution concentration estimates a cardiac output. In contrast to the isotonic injection, the hypertonic indicator interacts with the lung due to the osmotic pressure difference between the vascular blood vessel and lung. The blood osmolarity is quickly increased from the injected hypertonic solution, which generates the osmolarity imbalance between the blood vessel and lung. By the osmolarity equilibrium time tₑ, the lung water is transferred to the blood vessel due to the osmolarity imbalance. Right after the equilibrium time tₑ, the osmolarity is reversed, so the lung starts absorbing the water from the blood by the second osmolarity equilibrium. Movement of solutes, such as NaCl, is small enough to ignore relative to water exchange. Since a photoacoustic signal is affected by the amount of absorption of incident photons due to the hemoglobin concentration in blood, isotonic, hypertonic, and lung water contents in the blood vessel decrease the measured photoacoustic signal. In the disclosed example, these two base signals are set to be different for a general application. The most significant problem of these different baselines is that it is not straightforward to find the equilibrium time because the photoacoustic signal decreasing is started from different background.

The osmolarity (II) of the vascular blood vessels with the hypertonic injectate can be described as

$$Π ( t ) = ( {Π}_{h} - {Π}_{b} ) \frac{Δ {V}_{h}}{Δ V} \qquad ( 12 )$$

where Π_{b} and Πₕ are osmolarity of the pure blood and hypertonic solution, respectively, and are known. The amount of the lung water smeared into the blood vessel is omitted in Eq. (12) because the water transmittance is almost zero at the equilibrium time. Therefore, the osmolarity of the blood can be estimated at t=tₑ, from Eq. (13), which is the same as that in the lung at that time. At constant temperature, the volume of the EVLW, V_{LW}, can be estimated by

$${V}_{LW} = \vert \frac{Δ {V}_{LW}}{Δ {Π}_{L}} \vert {Π}_{L} \qquad ( 13 )$$

where ΔV_{LW}=ΔV_{LW}(tₑ)−ΔV_{LW}(t≦tᵢ)
and ΔΠ_{L}=Π_{L}(tₑ)−Π_{L}(t≦tᵢ) are the amount of differences of the lung volume and osmolarity, respectively. Π_{L}=Π_{L}(t≦tᵢ), which is also known. Note that the EVLW can be estimated once ΔV_{LW} and

$$\frac{Δ {V}_{h}}{Δ V}$$

in Eq. (12) are found. The baseline photoacoustic signal is

$${PA}_{b} = {Kc}_{tHb} + {PA}_{0} = K \frac{{tHb}_{b}}{Δ V} + {PA}_{0} \qquad ( 14 )$$

where C_{tHb} is a hemoglobin concentration in a unit volume ΔV before the injection. K is the conversion coefficient from C_{tHb} to a photoacoustic signal, which is assumed as constant during the indicator dilution measurement. The term PA₀ represents the photoacoustic signal from all photoacoustic sources insensitive to the indicator concentration change. It is assumed that PA₀ is the same for both dilution curves that is reasonable. After the hypertonic indicator injection, the photoacoustic signal becomes

PA^{h}(t)=Kc_{tHb}(t)+PA₀ (15)

where

c_{tHb}(t)=tHb_{b}/[ΔV_{b}(t)+ΔVₕ(t)+ΔV_{LW}(t)] (16)

In Eq. (6), ΔV_{b}(t), ΔVₕ(t), and ΔV_{LW}(t) indicate volumes of the blood, hypertonic, and lung water injected into the blood during dt, respectively. Since

$$\frac{{tHb}_{b}}{Δ V} = \frac{{tHb}_{b} ( t )}{Δ V ( t )}$$

Eq. (15) becomes

$$\frac{Δ {V}_{h} ( t )}{Δ V} + \frac{Δ {V}_{LW} ( t )}{Δ V} = \frac{1}{{α}^{h}} [ 1 - \frac{{PA}^{h} ( t )}{{PA}_{b}^{h}} ] \qquad ( 17 )$$

where α^{h}=(PA_{b}^{h}−PA₀)/PA_{b}^{h} that is always less than 1.
If the relationship between a photoacoustic signal and an isotonic dilution curve is considered on Eq. (17)

$$\frac{Δ {V}_{LW} ( t )}{Δ V} = \frac{1}{{α}^{h}} [ 1 - \frac{{PA}^{h} ( t )}{{PA}_{b}^{h}} ] - \frac{1}{{α}^{i}} [ 1 - \frac{{PA}^{i} ( t )}{{PA}_{b}^{i}} ] \qquad ( 18 )$$

where αⁱ=(PA_{b}ⁱ−PA₀)/PA_{b}ⁱ
that is known from a single isotonic curve. The superscripts h and i in Eq. (18) indicate hypertonic and isotonic solutions, respectively.

Under the assumption of that PA₀ is not changed, α^{h} can be found from PA_{b}^{h}. Also, at t=tₑ, ΔV_{LW}(tₑ)=0. Therefore,

$$\frac{Δ {V}_{h} ( {t}_{e} )}{Δ V}$$

in Eq. (13) can be calculated by

$$\frac{Δ {V}_{h} ( {t}_{e} )}{Δ V} = \frac{1}{{α}^{i}} [ 1 - \frac{{PA}^{i} ( {t}_{e} )}{{PA}_{b}^{i}} ] = \frac{1}{{α}^{h}} [ 1 - \frac{{PA}^{h} ( {t}_{e} )}{{PA}_{b}^{h}} ] \qquad ( 19 )$$

The lung volume change by t=tₑ is the equivalent to the amount of lung water smeared into the blood from the hypertonic injection time tᵢ to tₑ. If the time integration is applied to both sides of Eq. (18) using ΔV=F·dt,

$${\int }_{{t}_{i} = 0}^{{t}_{e}} Δ {V}_{LW} ( t ) d t = F \{ \frac{1}{{α}^{h}} {\int }_{0}^{{t}_{e}} [ 1 - \frac{{PA}^{i} ( t )}{{PA}_{b}^{i}} ] d t \} \qquad ( 20 )$$

where F is the blood flow rate. Therefore, from equations 19 and 20, the EVLW is Equation 13 is estimated using photoacoustic data. In this manner, the disclosed photoacoustic signal (i.e., the summed signal from the acoustic detector 20) may be used to provide an estimate of extravascular lung water.

The disclosed embodiments are provided in the context of indicator dilution curves. However, it should be understood that the disclosed techniques may be applied to other photoacoustic monitoring systems. Further, while the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A photoacoustic monitoring system, comprising:
a memory storing instructions for:
receiving a signal from an acoustic detector configured to detect a photoacoustic effect from light emitted into a patient's tissue, wherein the signal is representative of an indicator concentration in an artery and a vein; and
determining a physiological parameter based at least in part on the signal; and
a processor configured to execute the instructions.

2. The system of claim 1, wherein determining the physiological parameter based at least in part on the signal comprises providing a summed dilution curve based on the signal and determining the physiological parameter based on the summed dilution curve.

3. The system of claim 1, comprising a photoacoustic sensor comprising a light source and the acoustic detector, wherein the photoacoustic sensor is configured to generate the signal.

4. The system of claim 1, wherein the acoustic detector comprises a fixed focus ultrasound detector.

5. The system of claim 1, wherein the acoustic detector comprises a flat ultrasound detector.

6. The system of claim 4, wherein the fixed focus ultrasound detector has a central frequency of 1-5 MHz.

7. The system of claim 4, wherein the fixed focus ultrasound detector has a focal length of about 2 cm or less.

8. The system of claim 3, comprising an encoder disposed on the photoacoustic sensor or on a cable coupled to the photoacoustic sensor, wherein the encoder is configured to store one or more of calibration information or information related to characteristics of the acoustic detector.

9. The system of claim 1, wherein the physiological parameter comprises a cardiac output.

10. A photoacoustic monitoring system, comprising:
a monitor configured to:
receive a signal representative of an indicator concentration in an artery and a vein; and
determine a physiological parameter based at least in part on the signal; and
a sensor coupled to the monitor, comprising:
a light source configured to emit light into a tissue, wherein the tissue includes an artery and a vein; and
a flat or fixed focus ultrasound receiver configured to detect a photoacoustic effect from the light emitted into the tissue to generate the signal, wherein a focal length of the flat or fixed focus ultrasound receiver encompasses the artery and the vein.

11. The system of claim 10, wherein the focal length is about 7 cm or less.

12. The system of claim 10, wherein the focal length is between 0.1 cm and 2 cm.

13. The system of claim 10, wherein the monitor is configured to provide an indicator dilution curve from the signal.

14. The system of claim 10, wherein the physiological parameter is related to blood flow.

15. The system of claim 10, wherein the monitor is configured to receive an input related to an indicator injection time point.

16. A method, comprising:
using a processor:
receiving a signal from a fixed focus or flat acoustic detector configured to detect a photoacoustic effect from light emitted into a patient's tissue, wherein the signal is representative of an indicator concentration in an artery and a vein; and
determining a physiological parameter based at least in part on the signal.

17. The method of claim 16, comprising providing an indication of the physiological parameter on a display.

18. The method of claim 16, wherein determining the physiological parameter comprises determining an indicator concentration from the signal.

19. The method of claim 16, wherein the signal is representative of an indicator concentration in a temporal vein and artery.

20. The method of claim 16, wherein the signal is representative of an indicator concentration in a femoral vein and artery.
